(19) United States
(12) Patent Application Publication
(10) Pub. No.: US 2021/0162141 A1
(43) Pub. Date: Jun. 3, 2021

(54) ADMINISTRATION SYSTEM COMPRISING AN ADMINISTRATION DEVICE AND A PROTECTIVE DEVICE PROVIDED WITH A CLOSURE MEMBRANE

(71) Applicant: LABORATOIRE AGUETTANT, LYON (FR)

(72) Inventors: Cyril DODY, LYON (FR); Vincent GUYOT, MÜNCHEN (DE)

(21) Appl. No.: 17/263,478

(22) PCT Filed: Jul. 24, 2019
(86) PCT No.: PCT/FR2019/051838

§ 371 Date: Jan. 26, 2021

(30) Foreign Application Priority Data
Jul. 27, 2018 (FR) 18/57000

(51) Int. Cl. A61M 5/50; A61L 2/20

(52) U.S. Cl. CPC A61M 5/5086; A61M 2005/3104; A61L 2/20

(57) ABSTRACT

The administration system includes an administration device including a connection tip including a tubular connecting portion; and a protective device comprising a protective portion which is detachably connected to the administration device, a closing portion closing the free end of the tubular connecting portion, and a coupling portion rotatably coupled to the protective portion and to the closing portion. The protective device delimits an inner chamber within which the connection tip extends at least partially, and further includes at least one passage orifice opening into the inner chamber, and a closure membrane covering at least partially the at least one passage orifice.

### CROSS REFERENCE TO RELATED APPLICATIONS

This application is a National Stage of PCT Application No. PCT/FR2019/051838 filed on Jul. 24, 2019, which claims priority to French Patent Application No. 18/57000 filed on Jul. 27, 2018, the contents each of which are incorporated herein by reference thereto.

### TECHNICAL FIELD

The present invention concerns an administration system including an administration device and a protective device configured to protect at least partially a connection tip of the administration device.

### BACKGROUND

The document FR2896168 discloses an administration system including:
an administration device comprising a connection tip, the connection tip including a tubular connecting portion intended for the passage of a fluid, and a locking sleeve surrounding the tubular connecting portion and including an internal thread, and
a protective device delimiting at least partially an inner chamber within which the connection tip extends at least partially, the protective device comprising:
a protective portion which is detachably connected to the administration device, the protective portion covering at least partially and protecting at least partially the connection tip,
a closing portion tightly closing the free end of the tubular connecting portion of the connection tip,
a coupling portion rotatably coupled to the protective portion and to the closing portion such that a rotation of the protective portion about an axis of rotation substantially parallel to a longitudinal axis of the tubular connecting portion causes a rotation of the closing portion and a release of the free end of the tubular connecting portion, and
at least one passage orifice opening into the inner chamber and intended for the passage of a sterilization gas.

In particular, such a configuration of the administration system allows exposing the connection tip of the administration device to a sterilization gas, such as water steam, and therefore efficiently sterilizing the administration device.

Nonetheless, during the storage and/or handling of the administration system, impurities are possibly prone to penetrate into the inner chamber via the at least one passage orifice and therefore contaminate the connection tip.

In order to overcome this problem, it is known to dispose the administration system in a packaging, such as a blister pack provided with at least one wall permeable to the sterilization gas. However, the presence of such a packaging involves an additional opening step for the user.

### BRIEF SUMMARY

The present invention aims at overcoming all or part of these drawbacks.

Hence, the technical problem at the origin of the invention consists in providing an administration system that has a simple and economical structure, while enabling an optimum sterilization of the administration device and while guaranteeing the sterility of the connection tip of the administration device.

To this end, the present invention concerns an administration system, including:
an administration device comprising a connection tip, the connection tip including a tubular connecting portion intended for the passage of a fluid, and
a protective device delimiting at least partially an inner chamber within which the connection tip extends at least partially, the protective device comprising:
a protective portion which is detachably connected to the administration device, the protective portion covering at least partially and protecting at least partially the connection tip,
a closing portion tightly closing the free end of the tubular connecting portion of the connection tip,
a coupling portion rotatably coupled to the protective portion and to the closing portion such that a rotation of the protective portion about an axis of rotation substantially parallel to a longitudinal axis of the tubular connecting portion causes a rotation of the closing portion and a release of the free end of the tubular connecting portion, and
at least one passage orifice opening into the inner chamber,

characterized in that the coupling portion is distinct from the protective portion, in that the at least one passage orifice is provided on the coupling portion and in that the protective device further includes a closure membrane extending at least partially over the coupling portion and covering at least partially the at least one passage orifice.

Such a configuration of the administration system, and in particular the presence of the closure membrane, allows preventing the penetration of impurities into the inner chamber via the at least one passage orifice and therefore guaranteeing the sterility of the connection tip after the step of sterilizing the latter which may for example be carried out using a sterilization gas or also by irradiation, and in particular by gamma- or beta-ray irradiation.

The administration system may further have one or more of the following features, considered individually or in combination.

According to an embodiment of the invention, the at least one passage orifice is intended for the passage of a sterilization gas, and the closure membrane is porous and permeable to the sterilization gas. Such a configuration of the closure membrane enables the latter not to disturb the flow of a sterilization gas through the protective device and therefore the prior sterilization of the administration device using such a gas.

According to an embodiment of the invention, the closure membrane completely covers the at least one passage orifice.

According to an embodiment of the invention, the closure membrane tightly covers the at least one passage orifice.

According to an embodiment of the invention, the closure membrane is impermeable to the fluid.

According to an embodiment of the invention, the closure membrane is located upstream of the at least one passage orifice with respect to a flow direction of the sterilization gas through the protective device.

According to an embodiment of the invention, the protective portion surrounds at least partially the connection tip.

According to an embodiment of the invention, the closure membrane is impermeable to microorganisms.

According to an embodiment of the invention, the closure membrane is made of Tyvek (registered trademark) or of a paper that is porous and permeable to a sterilization gas.

According to an embodiment of the invention, the connection tip includes a locking sleeve surrounding the tubular connecting portion and including an internal thread.

According to an embodiment of the invention, the at least one passage orifice opens into the inner chamber opposite an annular volume delimited by the locking sleeve and the tubular connecting portion.

According to an embodiment of the invention, the closure membrane covers at least partially the coupling portion. Advantageously, the closure membrane bears against the coupling portion.

According to an embodiment of the invention, the protective device includes a plurality of passage orifices distributed, and for example evenly distributed, around the longitudinal axis of the tubular connecting portion.

According to an embodiment of the invention, the closure membrane is substantially planar.

According to an embodiment of the invention, the closure membrane is fastened to the protective portion and/or to the coupling portion, for example by heat-sealing.

According to an embodiment of the invention, the coupling portion includes first rotational coupling means configured to cooperate with second rotational coupling means provided on the protective portion.

According to an embodiment of the invention, the first rotational coupling means include a plurality of coupling projections provided on the coupling portion and angularly shifted from one another, and the second rotational coupling means include a plurality of coupling notches provided on the protective portion and angularly shifted from one another. Advantageously, the coupling projections extend substantially radially with respect to the longitudinal axis of the tubular connecting portion.

According to an embodiment of the invention, the coupling portion includes a central coupling housing within which the closing portion is mounted.

According to an embodiment of the invention, each of the central coupling housing and the closing portion has a non-circular, and for example hexagonal, cross-section.

According to an embodiment of the invention, the passage orifices are distributed around the central coupling housing.

According to an embodiment of the invention, the coupling portion and the closing portion are integrally made in one-piece. Advantageously, the coupling portion and the closing portion are formed by an insert interposed between the protective portion and the connection tip, and for example mounted on the protective portion. For example, the insert may be made of an elastomer.

According to an embodiment of the invention, the protective portion includes an inner housing within which the coupling portion is disposed.

According to an embodiment of the invention, the coupling portion includes a first surface directed towards the administration device and a second surface opposite to the first surface, the protective portion including an annular fastening surface which extends around the second surface of the coupling portion and which is substantially coplanar with the second surface of the coupling portion, the closure membrane extending partially over the second surface of the coupling portion and partially over the annular fastening surface.

According to an embodiment of the invention, the second surface of the coupling portion extends substantially perpendicular to the longitudinal axis of the tubular connecting portion.

According to an embodiment of the invention, the closing portion is connected by a frangible area to the free end of the tubular connecting portion of the connection tip.

According to an embodiment of the invention, the frangible area between the tubular connecting portion and the closing portion is made by an annular thinning of the material along the connecting line between the free end of the tubular connecting portion and the closing portion.

According to an embodiment of the invention, the coupling portion is rotatably coupled to the protective portion and to the closing portion such that a rotation of the protective portion about the axis of rotation causes a rotation of the closing portion and a break-up of the frangible area.

According to an embodiment of the invention, the protective device includes drive ribs formed on an outer surface of the protective portion, each drive rib extending substantially parallel to the longitudinal axis of the tubular connecting portion.

According to an embodiment of the invention, the protective portion includes a protective sleeve connected by a frangible connecting portion to the administration device.

According to an embodiment of the invention, the protective sleeve is coaxial with the tubular connecting portion.

Advantageously, the coupling portion is rotatably coupled to the protective portion and to the closing portion such that a rotation of the protective portion about the axis of rotation also causes a break-up of the frangible connecting portion.

According to an embodiment of the invention, the frangible connecting portion connects the protective sleeve to a body of the administration device from which the connection tip projects.

According to an embodiment of the invention, the protective sleeve surrounds the locking sleeve.

According to an embodiment of the invention, the frangible connecting portion connects the protective sleeve to a free end of the locking sleeve.

According to an embodiment of the invention, the frangible connecting portion includes a plurality of frangible connecting legs angularly spaced apart from one another with respect to the longitudinal axis of the tubular connecting portion. Advantageously, each connecting leg is substantially aligned longitudinally with a respective drive rib.

According to an embodiment of the invention, the frangible connecting portion is annular.

According to an embodiment of the invention, the frangible connecting portion between the protective sleeve and the administration device is made by an annular thinning of the material along the connecting line between the protective sleeve and the administration device.

According to another embodiment of the invention, the frangible connecting portion includes a tearable strip connecting the protective sleeve to the administration device, and more particularly to the body of the administration device. Advantageously, the tearable strip includes a gripping tab.

According to an embodiment of the invention, each drive rib is provided on an outer surface of the protective sleeve.

According to an embodiment of the invention, the at least one passage orifice opens opposite an annular volume delimited by the locking sleeve and the protective sleeve.

According to an embodiment of the invention, the coupling portion includes an outer coupling surface configured to cooperate, for example by friction, with an inner surface of the protective sleeve, and an inner coupling surface configured to cooperate, for example by friction, with an outer surface of the locking sleeve. Advantageously, the outer coupling surface of the coupling portion forms the first rotational coupling means, and the inner surface of the protective sleeve forms the second rotational coupling means.

According to an embodiment of the invention, the coupling portion includes at least one annular tightening rib provided on the inner coupling surface and configured to tightly cooperate with the outer surface of the locking sleeve.

According to an embodiment of the invention, the coupling portion extends within the annular volume delimited by the locking sleeve and the protective sleeve.

According to an embodiment of the invention, the coupling portion includes an anchorage portion configured to cooperate with the internal thread of the locking sleeve so as to block the translation of the coupling portion relative to the administration device. Advantageously, the anchorage portion is configured to enable an unscrewing of the coupling portion relative to the connection tip.

According to an embodiment of the invention, the closing portion includes a closing portion tightly closing the free end of the tubular connecting portion of the connection tip, and an annular tightening portion configured to tightly cooperate with an outer surface of the tubular connecting portion.

According to an embodiment of the invention, the protective portion includes a tubular wall projecting opposite to the administration device. Advantageously, the tubular wall is coaxial with the tubular connecting portion.

According to an embodiment of the invention, the protective sleeve has an outer diameter substantially equal to the outer diameter of the body of the administration device. According to a variant of the invention, the protective sleeve may have an outer diameter that is smaller than the outer diameter of the body of the administration device, and an inner diameter that is larger than or equal to the inner diameter of the locking sleeve. In addition, the outer diameter of the protective sleeve may be larger than the outer diameter of the locking sleeve.

According to an embodiment of the invention, the closing portion is made of an elastomer.

According to an embodiment of the invention, the closing portion is integrally made, and in particular molded, with the connection tip.

According to an embodiment of the invention, the coupling portion is made of a plastic material and for example of polypropylene.

According to an embodiment of the invention, the tubular connecting portion is truncated-cone shaped.

According to an embodiment of the invention, the locking sleeve is coaxial with the tubular connecting portion.

According to an embodiment of the invention, the connection tip of the administration device is of the Luer Lock, NR-Fit type or the same.

According to an embodiment of the invention, the protective portion is tubular.

According to an embodiment of the invention, the coupling portion is tubular.

According to an embodiment of the invention, the administration device is a pre-filled syringe.

According to an embodiment of the invention, the administration device is a pre-filled syringe, an empty syringe, a vial or an administration pouch, such as a perfusion pouch.

According to an embodiment of the invention, the protective portion is integrally made, and in particular molded, with the connection tip.

### BRIEF DESCRIPTION OF THE DRAWINGS

Anyway, the invention will be better understood from the following description with reference to the appended schematic drawings representing, as non-limiting examples, several embodiments of this administration system.

FIG. 1 is a partial perspective view of an administration system according to a first embodiment of the invention.

FIG. 2 is a partial longitudinal sectional view of the administration system of FIG. 1.

FIG. 3 is a partial truncated perspective view of the administration system of FIG. 1 in which the closure membrane has been removed.

FIG. 4 is a top view of the administration system of FIG. 1 in which the closure membrane has been removed.

FIG. 5 is a partial longitudinal sectional view of an administration system according to a second embodiment of the invention.

FIG. 6 is a partial longitudinal sectional view of an administration system according to a third embodiment of the invention.

FIG. 7 is a partial truncated perspective view of the administration system of FIG. 6 in which the closure membrane has been removed.

FIG. 8 is a partial longitudinal sectional view of an administration system according to a fourth embodiment of the invention.

FIG. 9 is a partial perspective view of the administration system of FIG. 8.

FIG. 10 is a top view of the administration system of FIG. 8 in which the closure membrane has been removed.

FIG. 11 is a longitudinal sectional view of an administration system according to a fifth embodiment of the invention.

### DETAILED DESCRIPTION

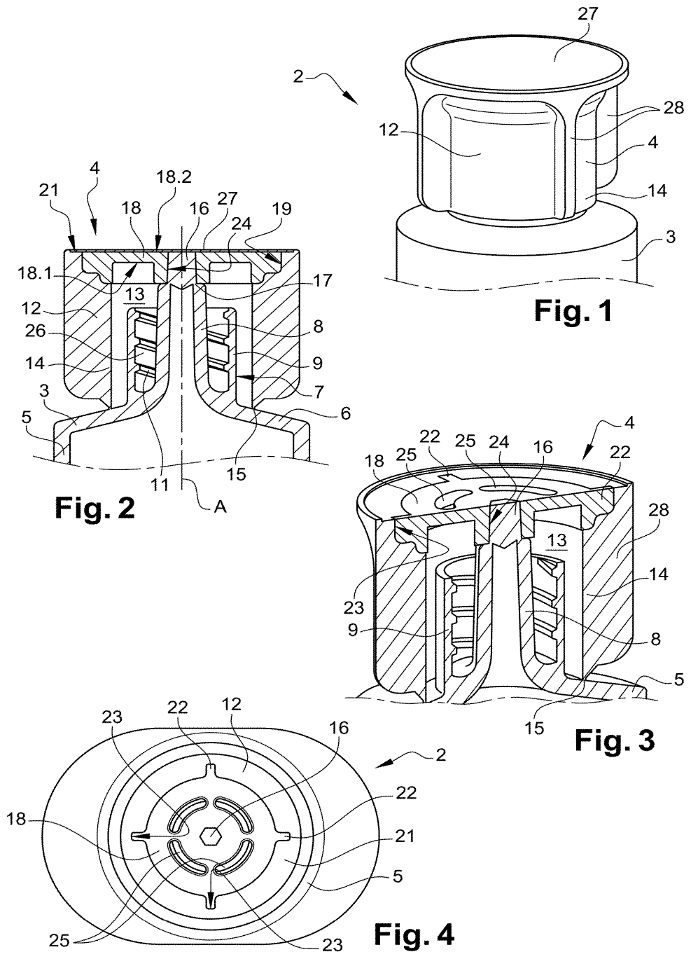

FIGS. 1 to 4 represent an administration system 2 according to a first embodiment of the invention, which comprises an administration device 3 and a protective device 4 mounted on the administration device 3.

According to this first embodiment of the invention, the administration device 3 is formed by a pre-filled syringe and comprises a body 5 with a generally cylindrical shape provided with a proximal end (not shown in FIGS. 1 to 4) and a proximal end 6. For example, the internal volume of the body 5 may be filled with a medicinal solution, a solvent, or any other fluid that could be delivered to a patient, or else be filled with a gas or with a solid, such as a powder and in particular a lyophilized product.

The administration device 3 also includes a connection tip 7 disposed at the level of the distal end 6 of the body 5. The connection tip 7 comprises a tubular connecting portion 8 fluidly connected to the internal volume of the body 5, and a locking sleeve 9 disposed coaxially with the tubular connecting portion 8 and surrounding the latter. Preferably, the locking sleeve 9 includes an internal thread 11, and the tubular connecting portion 8 is preferably truncated-cone shaped.

As shown more particularly in FIG. 2, the protective device 4 comprises a protective portion 12 which is tubular and which is detachably connected to the administration device 3. The protective portion 12 delimits at least partially an inner chamber 13 within which the connection tip 7 extends at least partially. Thus, the protective portion 12 covers at least partially and protects at least partially the connection tip 7.

According to the first embodiment of the invention, the protective portion 12 includes a protective sleeve 14 which surrounds the locking sleeve 9 and which is connected to the administration device 3, and more particularly to the body 5 of the administration device 3, by a frangible connecting portion 15. The frangible connecting portion 15 may be annular and be made for example by an annular thinning of the material along the connecting line between the protective sleeve 14 and the body 5 of the administration device 3. Advantageously, the protective sleeve 14 is coaxial with the tubular connecting portion 8, and has for example an inner diameter that is larger than the outer diameter of the locking sleeve 9 and smaller than the outer diameter of the body 5.

The protective device 4 further comprises a closing portion 16 tightly closing the free end of the tubular connecting portion 8 of the connection tip 7. According to the first embodiment of the invention, the closing portion 16 is integrally made with the connection tip 7, and is connected by a frangible area 17 to the free end of the tubular connecting portion 8 of the connection tip 7. For example, the frangible area 17 may be made by an annular thinning of the material along the connecting line between the free end of the tubular connecting portion 8 and the closing portion 16.

The protective device 4 also comprises a coupling portion 18 disposed within an inner housing 19 delimited by the protective portion 12, and more particularly by the protective sleeve 14. For example, the coupling portion 18 may be made of a plastic material and in particular of polypropylene.

According to the first embodiment of the invention, the coupling portion 18 has a disk-like general shape, and includes a first surface 18.1 directed towards the administration device 3 and a second surface 18.2 which is opposite to the first surface 18.1 and which is substantially planar. Advantageously, the second surface 18.2 of the coupling portion 18 extends substantially perpendicular to the longitudinal axis A of the tubular connecting portion 8, and the protective portion 12 includes an annular fastening surface 21 which extends around the second surface 18.2 of the coupling portion 18 and which is substantially coplanar with the second surface 18.2.

According to the first embodiment of the invention, the coupling portion 18 is distinct from the protective portion 12, and includes several coupling projections 22 (cf. FIGS. 3 and 4) angularly shifted from one another with respect to the longitudinal axis A and configured to cooperate with coupling notches 23 provided on the protective portion 12 and angularly shifted from one another with respect to the longitudinal axis A. Advantageously, the coupling projections 22 extend substantially radially with respect to the longitudinal axis A.

According to the first embodiment of the invention, the coupling portion 18 further includes a central coupling housing 24 within which the closing portion 16 is mounted. Advantageously, each of the central coupling housing 24 and the closing portion 16 has a non-circular, and for example hexagonal, cross-section.

Thus, the coupling portion 18 is rotatably coupled to the protective portion 12 and to the closing portion 16 such that a rotation of the protective portion 12, and more particularly of the protective sleeve 14, about an axis of rotation parallel to the longitudinal axis A of the tubular connecting portion 8 causes a rotation of the closing portion 16 and a break-up on the one hand of the frangible connecting portion 15 and, on the other hand, of the frangible area 17.

The protective device 4 further includes several passage orifices 25 provided on the coupling portion 18 and opening into the inner chamber 13. More particularly, each passage orifice 25 is intended for the passage of a sterilization gas which may, for example, consist of water steam or of ethylene oxide. Advantageously, the passage orifices 25 are evenly distributed around the central coupling housing 24, and therefore evenly distributed around the longitudinal axis A of the tubular connecting portion 8. According to the first embodiment of the invention, each passage orifice 25 opens into the inner chamber 13 at least partially opposite an annular volume 26 (cf. FIG. 3) delimited by the locking sleeve 9 and the tubular connecting portion 8. Nonetheless, each passage orifice 25 may also open into the inner chamber 13 at least partially opposite an annular volume delimited by the locking sleeve 9 and the protective sleeve 14.

In addition, the protective device 4 includes a closure membrane 27 which is substantially planar and which is located upstream of the passage orifices 25 with respect to the flow direction of the sterilization gas through the protective device 4. The closure membrane 27 is porous and permeable to the sterilization gas. Advantageously, the closure membrane 27 is impermeable to microorganisms, and is made for example of Tyvek (registered trademark).

According to the first embodiment of the invention, the closure membrane 27 covers the coupling portion 18 and in particular the passage orifices 25, and is fastened to the annular fastening surface 21 of the protective portion 12.

According to the first embodiment of the invention, the protective device 4 also includes several drive ribs 28 which extend parallel to the longitudinal axis A of the tubular connecting portion 8, and which are formed on an outer surface of the protective portion 12, and more particularly of the protective sleeve 14. Such drive ribs 28 facilitate the rotational driving of the protective portion 12 by a user, and also prevents the latter from attempting to separate the protective device 4 from the administration device 3 by pulling or bending. Advantageously, the drive ribs 28 are evenly distributed around the longitudinal axis A. Nonetheless, the protective device 4 may be devoid of any drive ribs 28.

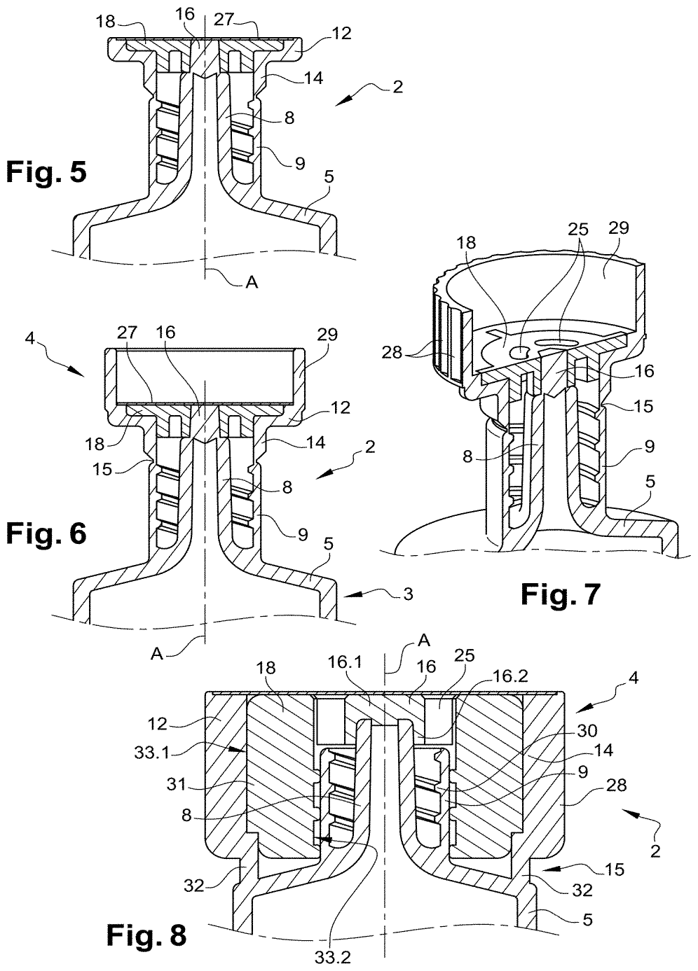

FIG. 5 represents an administration system 2 according to a second embodiment of the invention which differs from the first embodiment represented in FIGS. 1 to 4 mainly in that the frangible connecting portion 15 connects the protective sleeve 14 to a free end of the locking sleeve 9. According to such an embodiment of the invention, the protective sleeve 14 has an inner diameter corresponding substantially to the inner diameter of the locking sleeve 9.

FIGS. 6 and 7 represent an administration system 2 according to a third embodiment of the invention which differs from the second embodiment represented in FIG. 5 mainly in that the protective portion 12 includes a tubular portion 29 which projects opposite to the administration device 3 and which is coaxial with the protective sleeve 14. Advantageously, the tubular wall 29 projects with respect to the closure membrane 27.

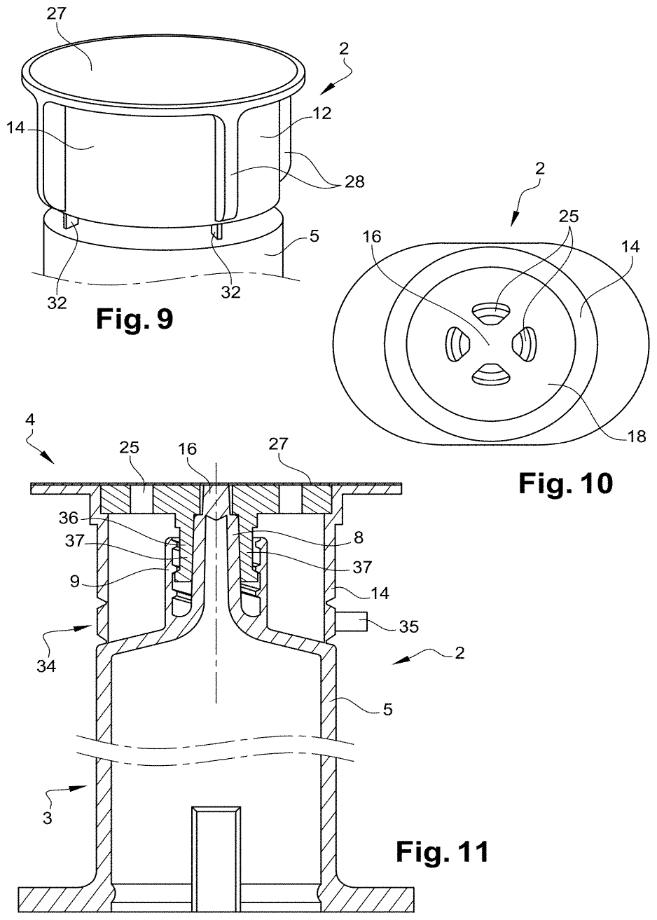

FIGS. 8 to 10 represent an administration system 2 according to a fourth embodiment of the invention which differs from the first embodiment represented in FIGS. 1 to 4 mainly in that the protective device 4 includes an insert 31, made for example of an elastomer, fastened to the protective portion 12 and forming the coupling portion 18 and the closing portion 16. Thus, according to such an embodiment of the invention, the coupling portion 18 and the closing portion 16 are integrally made in one-piece.

Furthermore, according to the fourth embodiment of the invention, the frangible connecting portion 15 includes a plurality of frangible connecting legs 32 angularly spaced apart from one another with respect to the longitudinal axis A of the tubular connecting portion 8. Advantageously, each connecting leg 32 is substantially aligned longitudinally with a respective drive rib 28.

In addition, according to the fourth embodiment of the invention, the coupling portion 18 extends within an annular volume delimited by the locking sleeve 9 and the protective sleeve 14, and includes an outer coupling surface 33.1 configured to cooperate, for example by friction, with an inner surface of the protective sleeve 14, and an inner coupling surface 33.2 configured to cooperate, for example by friction, with an outer surface of the locking sleeve 9. Advantageously, the coupling portion 18 includes several annular tightening ribs 30 provided on the inner coupling surface 33.2 and shifted according to the longitudinal axis A of the tubular connecting portion 8. Such annular tightening ribs 30 are configured to tightly cooperate with the outer surface of the locking sleeve and thus form a sterile barrier which prevents the passage of impurities in the direction of the free end of the connection tip 7.

The fourth embodiment of the invention also differs from the first embodiment of the invention in that the closing portion 16 includes a closing portion 16.1 tightly closing the free end of the tubular connecting portion 8 of the connection tip 7, and an annular tightening portion 16.2 which extends along an outer surface of the tubular connecting portion 8, the annular tightening portion 16.2 being configured to tightly cooperate with the outer surface of the tubular connecting portion 8.

FIG. 11 represents an administration system 2 according to a fifth embodiment of the invention which differs from the first embodiment represented in FIGS. 1 to 4 mainly in that the frangible connecting portion 15 includes a tearable strip 34 connecting the protective sleeve 14 to the administration device 3, and more particularly to the body 5 o the administration device 3. Advantageously, the tearable strip 34 includes a gripping tab 35.

According to the fifth embodiment of the invention, the coupling portion 18 further includes an anchorage portion 36 configured to cooperate with the internal thread 11 of the locking sleeve 9 so as to block the translation of the coupling portion 18 relative to the administration device 3. Advantageously, the anchorage portion 36 projects in the direction of the administration device 3 and is configured to enable an unscrewing of the coupling portion 18 relative to the connection tip 7. For example, the anchorage portion 36 may be formed by several anchorage tabs 37 extending substantially parallel to the longitudinal axis A of the tubular connecting portion 8, the anchorage tabs being distributed around the tubular connecting portion 8.

According to a variant which is not represented in the figures, the closing portion 16 may be distinct from the coupling portion 18 and from the connection tip 7, made of an elastomer and attached and fastened on the coupling portion 18 so as to be rotatably coupled to the latter.

It goes without saying that the invention is not limited to the sole embodiments of this administration system, described hereinabove as examples, it comprises, on the contrary, all variants thereof. Thus, in particular, the step of sterilizing the administration device may be carried out by irradiation, and in particular by gamma- or beta-ray irradiation.

1. An administration system, including:
an administration device comprising a connection tip, the connection tip including a tubular connecting portion intended for the passage of a fluid, and
a protective device delimiting at least partially an inner chamber within which the connection tip extends at least partially, the protective device comprising:
a protective portion which is detachably connected to the administration device, the protective portion covering at least partially and protecting at least partially the connection tip,
a closing portion tightly closing a free end of the tubular connecting portion of the connection tip,
a coupling portion rotatably coupled to the protective portion and to the closing portion such that a rotation of the protective portion about an axis of rotation substantially parallel to a longitudinal axis of the tubular connecting portion causes a rotation of the closing portion and a release of the free end of the tubular connecting portion, and
at least one passage orifice opening into the inner chamber,
wherein the coupling portion is distinct from the protective portion, in that the at least one passage orifice is provided on the coupling portion and in that the protective device further includes a closure membrane extending at least partially over the coupling portion and covering at least partially the at least one passage orifice.

2. The administration system according to claim 1, wherein the at least one passage orifice is intended for the passage of a sterilization gas, and the closure membrane is porous and permeable to the sterilization gas.

3. The administration system according to claim 1, wherein the closure membrane is made of Tyvek or of a paper that is porous and permeable to a sterilization gas.

4. The administration system according to claim 1, wherein the coupling portion includes first rotational coupling means configured to cooperate with second rotational coupling means provided on the protective portion.

5. The administration system according to claim 1, wherein the coupling portion includes a central coupling housing within which the closing portion is mounted.

6. The administration system according to claim 1, wherein the coupling portion and the closing portion are integrally made in one-piece.

7. The administration system according to claim 1, wherein the protective portion includes an inner housing within which the coupling portion is disposed.

8. The administration system according to claim 1, wherein the coupling portion includes a first surface directed towards the administration device and a second surface opposite to the first surface, the protective portion including an annular fastening surface which extends around the second surface of the coupling portion and which is substantially coplanar with the second surface of the coupling portion, the closure membrane extending partially over the second surface of the coupling portion and partially over the annular fastening surface.

9. The administration system according to claim 1, wherein the closing portion is connected by a frangible area to the free end of the tubular connecting portion of the connection tip.

10. The administration system according to claim 1, wherein the protective device includes drive ribs formed on an outer surface of the protective portion, each drive rib extending substantially parallel to the longitudinal axis of the tubular connecting portion.

11. The administration system according to claim 1, wherein the connection tip includes a locking sleeve surrounding the tubular connecting portion and including an internal thread.

12. The administration system according to claim 1, wherein the protective portion includes a protective sleeve connected by a frangible connecting portion to the administration device.

13. The administration system according to claim 12, wherein the frangible connecting portion connects the protective sleeve to a body of the administration device from which the connection tip projects.

14. The administration system according to claim 12, wherein the protective sleeve surrounds the locking sleeve.

15. The administration system according to claim 12, wherein the frangible connecting portion connects the protective sleeve to a free end of the locking sleeve.

16. The administration system according to claim 1, wherein the administration device is one of; a pre-filled syringe, an empty syringe, a vial or an administration pouch.

17. The administration system according to claim 2, wherein the closure membrane is made of Tyvek or of a paper that is porous and permeable to a sterilization gas.

18. The administration system according to claim 17, wherein the coupling portion includes first rotational coupling means configured to cooperate with second rotational coupling means provided on the protective portion.

19. The administration system according to claim 18, wherein the coupling portion includes a central coupling housing within which the closing portion is mounted.

20. The administration system according to claim 19, wherein the coupling portion and the closing portion are integrally made in one-piece.
